(19) United States
(12) Patent Application Publication
(10) Pub. No.: US 2020/0129486 A1
(43) Pub. Date: Apr. 30, 2020

(54) METHODS AND PHARMACEUTICAL COMPOSITIONS FOR THE TREATMENT OF OLMSTED SYNDROME

(71) Applicants: INSERM (Institut National de la Santé et de la Recherche Medicale), Paris (FR); Université Paris Descartes, Paris (FR); Assistance Publique-Hôpitaux de Paris (APHP), Paris (FR); Fondation Imagine, Paris (FR)

(72) Inventors: Alain HOVNANIAN, Paris (FR); Sabine DUCHATELET, Paris (FR)

(21) Appl. No.: 16/626,370

(22) PCT Filed: Jun. 25, 2018
(86) PCT No.: PCT/EP2018/066871

§ 371 Date: Dec. 24, 2019

(30) Foreign Application Priority Data
Jun. 26, 2017 (EP) 17305790.2

(51) Int. Cl. A61K 31/436; A61K 9/00; A61P 17/12

(52) U.S. Cl. CPC A61K 31/436; A61P 17/12; A61K 9/0014

(57) ABSTRACT

Olmsted syndrome (OS) is a rare genodermatosis. The disease is debilitating and progressive keratoderma and auto-amputation of digits can prevent patients from grasping and walking, and confine them to a wheelchair. New therapeutic options are therefore crucial and are expected from a better understanding of the disease mechanisms. The inventors show an abnormal mTOR pathway activation in OS lesional skin. Topical treatment with 1% Sirolimus shows good tolerance and partial but real efficacy on budding, inflammatory and hyperkeratotic lesions of the sole was observed in the treated patient. Accordingly, the present invention relates to a method of treating Olmsted syndrome in a patient in need thereof comprising administering to the patient a therapeutically effective amount of an mTOR inhibitor.

### FIELD OF THE INVENTION

The present invention relates to methods and pharmaceutical compositions for the treatment of Olmsted syndrome.

### BACKGROUND OF THE INVENTION

Olmsted syndrome (OS) is a rare genodermatosis classically characterized by the combination of bilateral mutilating transgredient palmoplantar keratoderma (PPK) and periorificial keratotic plaques, but which shows considerable clinical heterogeneity. The disease starts usually at birth or in early childhood. About 73 cases have been reported worldwide. OS is observed in both sexes, although male cases are more frequent. The most suggestive symptoms associate PPK with pseudoainhum and periorificial keratotic plaques. Frequently associated features include hair and nail abnormalities, leukokeratosis, corneal default and recurrent infections. Pain and itching are variable but can be severe. Most of reported OS cases are sporadic, although familial cases with different mode of inheritance were also described. Mutations in TRPV3 (Transient receptor potential vanilloid-3) gene have recently been identified as a cause of autosomal dominant (gain-of-function mutations) or recessive OS. Mutations in MBTPS2 (membrane-bound transcription factor protease, site 2) gene were identified in a recessive X-linked form. The diagnosis relies mainly on clinical features associating severe PPK and periorificial keratotic plaques, but can be challenging in patients with incomplete phenotype or atypical features. OS has to be differentiated from other severe forms of PPK including Vohwinkel, Clouston, Papillon-Lefèvre or Haim-Munk syndromes, Mal de Meleda, pachyonychia congenita, Tyrosinemia type II and acrodermatitis enteropathica. When differential diagnoses are difficult to exclude, genetic studies are essential to search for a TRPV3 or MBTPS2 mutation. However, additional genes remain to be identified. No specific and satisfactory therapy is currently available for OS. Current treatments of hyperkeratosis (mainly emollients, keratolytics, retinoids or corticosteroids), either topical or systemic, are symptomatic and offer only temporary partial relief. Specific management of pain and itching is important to reduce the morbidity of the disease. The disease is debilitating and progressive keratoderma and auto-amputation of digits can prevent patients from grasping and walking, and confine them to a wheelchair. New therapeutic options are therefore crucial and are expected from a better understanding of the disease mechanisms.

### SUMMARY OF THE INVENTION

The present invention relates to methods and pharmaceutical compositions for the treatment of Olmsted syndrome. In particular, the present invention is defined by the claims.

### DETAILED DESCRIPTION OF THE INVENTION

The first object of the present invention relates to a method of treating Olmsted syndrome in a patient in need thereof comprising administering to the patient a therapeutically effective amount of an mTOR inhibitor.

As used herein, the term “Olmsted syndrome” has its general meaning in the art and refers to a hereditary palmoplantar keratoderma characterized by the combination of bilateral mutilating transgredient palmoplantar keratoderma and periorificial keratotic plaques. The term is also known as Mutilating palmoplantar keratoderma (PPK) with periorificial keratotic plaques. The disease starts usually at birth, in neonatal period or in early childhood, when the child starts to walk and grasp, and worsens over time. The disease has a slow but progressive course.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “mTOR inhibitor” refers to a compound (natural or synthetic) that inhibits at least one activity of an mTOR, such as the serine/threonine protein kinase activity on at least one of its substrates (e.g., p70 S6 kinase 1, 4E-BP1, AKT/PKB and eEF2). A person skilled in the art can readily determine whether a compound, such as rapamycin or an analogue or derivative thereof, is an mTOR inhibitor. A specific method of identifying such compounds is disclosed in U.S. Patent Application Publication No. 2003/0008923.

In some embodiments, the mTOR inhibitor is selected from the group consisting of rapamycin (also called sirolimus and described in U.S. Pat. No. 3,929,992), temsirolimus, deforolimus, everolimus, tacrolimus and rapamycin analogue or derivative thereof.

As used herein, the term “rapamycin analogue or derivative thereof” includes compounds having the rapamycin core structure as defined in U.S. Patent Application Publication No. 2003/0008923 (which is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus. Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). Examples of 27-esters and ethers of rapamycin are disclosed in U.S. Pat. No. 5,256,790, which is hereby incorporated by reference in its entirety. Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference. Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein. Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC and PROGRAF. Other preferred mTOR inhibitors include AP-23675, AP-23573, and AP-23841 (Ariad Pharmaceuticals). Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxyl)ethyl rapamycin (U.S. Pat. No. 5,665,772).

By a “therapeutically effective amount” is meant a sufficient amount of the mTOR inhibitor to treat Olmsted syndrome at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the agent, preferably from 1 mg to about 100 mg of the agent. An effective amount of the drug is ordinarily supplied at a dosage level from 0.0002 mg/kg to about 20 mg/kg of body weight per day, especially from about 0.001 mg/kg to 7 mg/kg of body weight per day.

Typically the mTOR inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils.

In some embodiments, it may be desirable to administer the mTOR inhibitor of the present in a topical formulation. As used herein the term “topical formulation” refers to a formulation that may be applied to skin. Topical formulations can be used for both topical and transdermal administration of substances. As used herein, “topical administration” is used in its conventional sense to mean delivery of a substance, such as a therapeutically active agent, to the skin or a localized region of a subject's body. As used herein, “transdermal administration” refers to administration through the skin. Transdermal administration is often applied where systemic delivery of an active is desired, although it may also be useful for delivering an active to tissues underlying the skin with minimal systemic absorption. Typically, the topical pharmaceutically acceptable carrier is any substantially nontoxic carrier conventionally usable for topical administration of pharmaceuticals in which the mTOR inhibitor of the present invention will remain stable and bioavailable when applied directly to skin surfaces. For example, carriers such as those known in the art effective for penetrating the keratin layer of the skin into the stratum comeum may be useful in delivering the mTOR inhibitor of the present invention to the area of interest. Such carriers include liposomes. mTOR inhibitor of the present invention can be dispersed or emulsified in a medium in a conventional manner to form a liquid preparation or mixed with a semi-solid (gel) or solid carrier to form a paste, powder, ointment, cream, lotion or the like. Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy. The topical acceptable carrier will be any substantially non-toxic carrier conventionally usable for topical administration in which mTOR inhibitor of the present invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Any suitable carrier or vehicle effective for topical administration to a patient as know in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. Topical acceptable carriers may be similar or identical in nature to the above described topical pharmaceutically acceptable carriers. It may be desirable to have a delivery system that controls the release of mTOR inhibitor of the present invention to the skin and adheres to or maintains itself on the skin for an extended period of time to increase the contact time of the mTOR inhibitor of the present invention on the skin. Sustained or delayed release of mTOR inhibitor of the present invention provides a more efficient administration resulting in less frequent and/or decreased dosage of mTOR inhibitor of the present invention and better patient compliance. Examples of suitable carriers for sustained or delayed release in a moist environment include gelatin, gum arabic, xanthane polymers. Pharmaceutical carriers capable of releasing the mTOR inhibitor of the present invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the mTOR inhibitor of the present invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The mTOR inhibitor of the present invention can also be administered in combination with other pharmaceutically effective agents including, but not limited to, antibiotics, other skin healing agents, and antioxidants. In some embodiments, the topical formulation of the present invention comprises a penetration enhancer. As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

The invention will be further illustrated by the following figures and examples. However, these examples and figures should not be interpreted in any way as limiting the scope of the present invention.

### FIGURES

FIG. 1. pRPS6 immunostaining on skin from healthy controls.

FIG. 2. pRPS6 immunohistostaining on lesional skin from OS patients.

FIG. 3. pRPS6 study by western-blot

### EXAMPLE 1

Material & Method

mTor signaling activates S6 kinase, which phosphorylates Ser235, Ser236, Ser240 and Ser244 of the ribosomal protein S6 (RPS6). Healthy skin and lesional skin from five OS patients were investigated by immunostaining using an anti pSer240/244 RPS6 antibody (#5364 Cell Signaling). In addition, p-RPS6 was investigated in cultured primary keratinocytes (proliferative conditions) from healthy individuals and from OS patient C by western-blot analysis (#5364 Cell Signaling).

Results

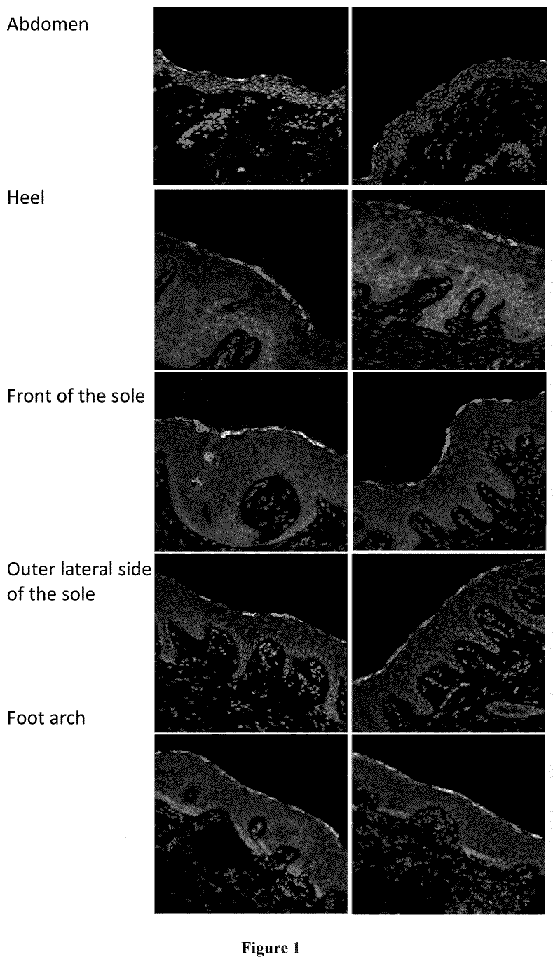

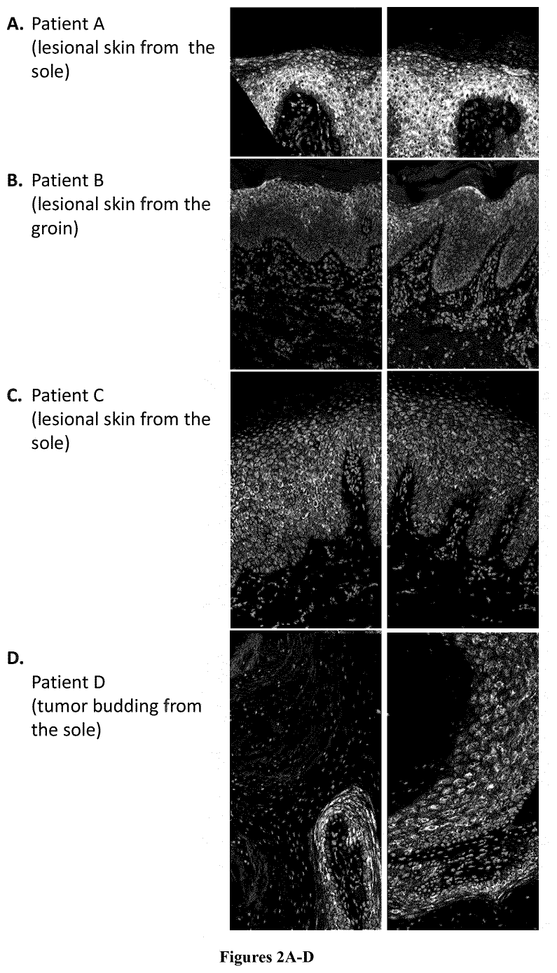

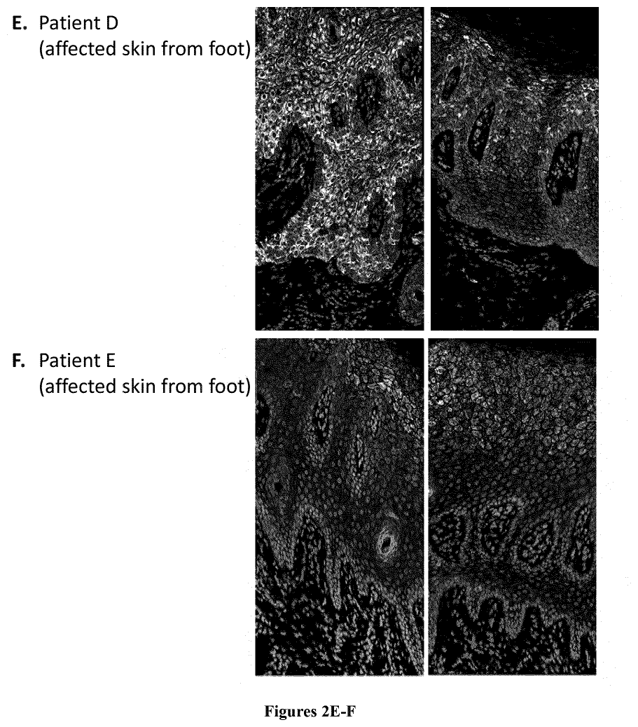

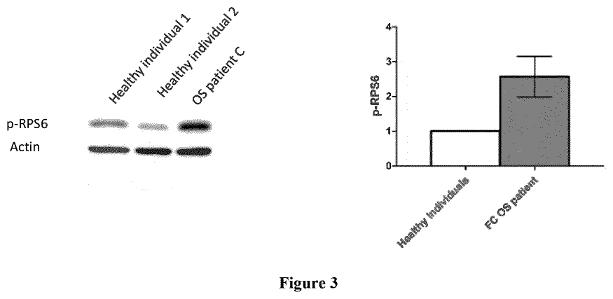

Staining was restricted to the stratum granulosum in abdominal skin from healthy controls, whereas staining extended to all layers of the epidermis from the sole of a healthy control although weaker (FIG. 1). Increased staining extending to all epidermal layers was observed in OS lesional skin mainly in patients A to D (FIG. 2). Increased RPS6 phosphorylation indicates abnormal mTOR pathway activation in OS lesional skin. In addition, increased p-RPS6 levels in OS patient cultured keratinocytes provide evidence for enhanced mTOR pathway activation in steady-state conditions (FIG. 3).

### EXAMPLE 2

I—Clinical Observation

The patient is an 11 year-old girl who displayed at birth superficial peeling of her toes without hyperkeratosis, nor blistering of her skin.

Plantar keratoderma developed after she started walking at 1 year of age. It was initially distributed to islands on pressure points but gradually extended to the entire plantar surface. Plantar skin became hyperproliferative and budding, covered with dramatical hyperkeratosis overtime.

Plantar keratoderma was associated with intense erythermalgia diagnosed at 3 years, manifesting by severe itch, burning pain, erythema and warmth in the extremities (hands, feet and ears), and venous dilatation.

Because of extreme foot pain, she walked on knees and hands which resulted in localized palmar keratoderma, and has been using a wheelchair since the age of 3.

Her finger and toe nails were thin and brittle.

Her hair was fine, dry, curly and unmanageable.

We confirmed the diagnosis of severe Olmsted syndrome by identifying a missense c.2017C>T (p.Leu673Phe) mutation in TRPV3 present at the heterozygous in the patient. None of her parents had this mutation.

II—Off-Label Use of 1% Topical Sirolimus—Design

GMP Grade Sirolimus
from accredited distributor (Pharmacie des Hôpitaux de Paris)
1% formulation in Dexeryl cream (emollient)
1 g (1,250 euros)
100 g preparation at 1%
1 month stability
Kept at +4° C.

Dose:

3 grams of the preparation at 1% applied daily on the right sole by the patient, with a disposable glove

The corresponding amount was initially weighed on a scale

Duration:

Initially planned for 4 months

Start: Nov. 3, 2015

End: Feb. 5, 2016 (due to the development of an abscess of the inner lateral side of treated foot, distant from the sole)

Tolerance:

Phone contact every 2 weeks

Monthly routine laboratory tests

Efficacy:

Daily pain diary rating the level of pain on a scale from 0 to 10 at least 2 different times of the day

Validated life quality evaluation (DLQI) every 2 weeks

Clinical pictures every 2 weeks

Clinical examination every month

Safety:

Monthly sirolimus levels in the serum (therapeutic ranges 9-12 ng/ml)

III—Results:

Tolerance:

Tolerance was good, with no obvious side effects. Occurrence of an abcess of the right foot due to Staph. aureus at the end of the study (M3), but distant from the treated area (inner lateral side of the right foot)

Efficacy:

Daily rating of the pain level remained surprisingly low (2-3), with several scores of 1 during M2 and M3. Life quality evaluation (DLQI) remained almost unchanged.

Clinic examination showed partial reduction of hyperkeratosis, of skin inflammation and of budding as evidenced by clinical pictures in January 2016 (M2). In particular, we observed a good clinical response one month following the start of topical treatment on the right sole. Hyperkeratosis, budding and inflammation were reduced. Then, rapid aggravation was observed one month after stopping topical treatment on the right sole: massive hyperkeratosis, budding and inflammation reappeared.

Safety:

Sirolimus levels in the serum remained below therapeutic ranges (2.1 ng/ml)

IV—Conclusion

The mTOR pathway is activated in this OS patient (increased phosphorylation of RPS6 on skin sections).

Topical treatment with 1% Sirolimus shows good tolerance.

Partial but real efficacy on budding, inflammatory and hyperkeratotic lesions of the sole was observed.

No striking effect on pain level nor on quality of life index.

No significant systemic passage of the drug.

### REFERENCES

Throughout this application, various references describe the state of the art to which this invention pertains. The disclosures of these references are hereby incorporated by reference into the present disclosure.

1. A method of treating Olmsted syndrome in a patient in need thereof comprising administering to the patient a therapeutically effective amount of an mTOR inhibitor.

2. The method of claim 1 wherein the mTor inhibitor is selected from the group consisting of sirolimus, temsirolimus, deforolimus, everolimus, tacrolimus and rapamycin analogue or derivative thereof.

3. The method of claim 1 wherein the mTOR inhibitor is administered to the patient with a topical formulation.
